(19) United States
(12) Patent Application Publication
(10) Pub. No.: US 2024/0156755 A1
(43) Pub. Date: May 16, 2024

(54) USE OF RIP1 INHIBITOR OR MLKL INHIBITOR FOR TREATING OR PREVENTING HEREDITARY RETINAL DYSTROPHY

(71) Applicant: TZU CHI UNIVERSITY, HUALIEN CITY (TW)

(72) Inventors: Shun-Ping HUANG, HUALIEN CITY (TW); Jia-Ying CHIEN, HUALIEN CITY (TW)

(21) Appl. No.: 18/085,119

(22) Filed: Dec. 20, 2022

(30) Foreign Application Priority Data
Nov. 2, 2022 (TW) 111141817

(51) Int. Cl. A61K 31/165; A61K 31/505; A61K 48/00; A61P 27/02

(52) U.S. Cl. CPC A61K 31/165; A61K 31/505; A61K 48/0016; A61P 27/02

(57) ABSTRACT

The present invention discloses a method for treating or preventing hereditary retinal dystrophy, the method comprising: administering a composition comprising a RIP1 inhibitor or a MLKL inhibitor to a subject in need thereof. The RIP1 inhibitor is, for example, RIPA-56, and the MLKL inhibitor is, for example, GW806742X.

### FIELD OF THE INVENTION

The present invention is related to pharmaceutical use of a RIP1 inhibitor or a MLKL inhibitor, and particularly to, use of a RIP1 inhibitor or a MLKL inhibitor for treating or preventing hereditary retinal dystrophy.

### BACKGROUND OF THE INVENTION

Hereditary retinal dystrophy (HRD) refers to a group of genetic retinal disorders exhibiting both genetic heterogeneity and phenotypic heterogeneity, such as retinitis pigmentosa (RP), Leber's congenital amaurosis (LCA), Usher syndrome, choroideremia, retinoschisis, and Stargardt disease. HRD remains major obstacles in ophthalmology despite technology that has changed the scope of medicine. To date, more than 280 genes associated with HRD have been reported. However, there is still no curative therapy for HRD at present. The dilemma in understanding HRD is the question how so many different and diverse primary genetic lesions cause the same clinical manifestation which characterizes HRD.

Gene therapy has emerged after the discovery of DNA. However, the treatment is still unsuccessful because of limited knowledge of gene expression and the selection of administering genetic material. Currently, gene therapy in various diseases has been initiated. The eye is a general focus of gene therapy because it is a small and enclosed organ with unique immunological properties. Furthermore, the noninvasive imaging system can be used for treatment results compared with untreated eyes. When the mutant gene associated with HRD is identified, gene therapy is proven effective in clinical development. Gene therapy produces the somatic cells of patients to generate specific therapeutic proteins for the modulation of genetic diseases.

For the reason that the knowledge in gene therapy is inadequate for the current medical science, there is a need to find a molecule drug for treating HRD.

### SUMMARY OF THE INVENTION

An objective of the present invention is to provide a method for treating or preventing hereditary retinal dystrophy, the method comprising: administering a composition comprising a RIP1 inhibitor or a MLKL inhibitor to a subject in need thereof.

Exemplarily, the RIP1 inhibitor is RIPA-56.

Exemplarily, the MLKL inhibitor is GW806742X.

Exemplarily, the hereditary retinal dystrophy is hereditary retinal dystrophy caused by Pomgnt1 mutation.

Exemplarily, the hereditary retinal dystrophy caused by Pomgnt1 mutation is hereditary retinal dystrophy caused by Pomgnt1^{L120R/L120R} mutation.

Exemplarily, the hereditary retinal dystrophy comprises: retinitis pigmentosa, Leber's congenital amaurosis, Usher syndrome, choroideremia, retinoschisis, or Stargardt disease.

Exemplarily, the composition is provided for lowering the expression level of beclin1, P62, or LC3B.

Exemplarily, the composition is provided for enhancing the trans-epithelial electrical resistance of retinal pigment epithelial cells.

Exemplarily, the composition administering step is performed in combination with gene therapy.

Exemplarily, the gene therapy comprises: administering a nucleic acid for expressing protein POMGNT1 to the subject.

### BRIEF DESCRIPTION OF THE DRAWINGS

FIG. 1A is a scotopic electroretinogram showing the a- and b-waves of Pomgnt1^{L120R/L120R} mice in different age group;

FIG. 1B is a line graph comparing the a- and b-wave amplitudes of Pomgnt1^{L120R/L120R} mice in different age group;

FIG. 1C is a scotopic electroretinogram showing the c-waves of Pomgnt1^{L120R/L120R} mice in different age group;

FIG. 1D is a line graph comparing the c-wave amplitudes of Pomgnt1^{L120R/L120R} mice in different age group;

FIG. 2A is a scotopic electroretinogram showing the a- and b-waves of Pomgnt1^{L120R/L120R} mice after 3 months' treatment and 6 months' treatment;

FIG. 2B is a line graph comparing the a- and b-wave amplitudes of Pomgnt1^{L120R/L120R} mice after 3 months' treatment;

FIG. 2C is a line graph comparing the a- and b-wave amplitudes of Pomgnt1^{L120R/L120R} mice after 6 months' treatment;

FIG. 3A is an immunoblotting picture showing the protein expression in the retina of a Pomgnt1^{L120R/L120R} mouse;

FIG. 3B is a bar graph comparing the relative protein expression level in the retina of a Pomgnt1^{L120R/L120R} mouse;

FIG. 4 is a schematic diagram showing the mechanism of neuron death in a Pomgnt1^{L120R/L120R} mouse;

FIG. 5 is a fluorescence microscopic picture showing the AAV transduction result in cells;

FIG. 6 is a bar graph showing the trans-epithelial electrical resistance of cells after AAV transduction;

FIG. 7A is a confocal microscopic picture showing the status of Golgi complexes in cells after AAV transduction;

FIG. 7B is a bar graph showing the fragmentation level of Golgi complexes in cells after AAV transduction;

FIG. 8 is a bar graph showing the trans-epithelial electrical resistance of cells after RIPA-56 treatment or GW806742X treatment;

FIG. 9A is an immunoblotting picture showing the protein expression in cells after RIPA-56 treatment; and

FIG. 9B is a bar graph comparing the relative protein expression level in cells after RIPA-56 treatment.

### DETAILED DESCRIPTION OF THE INVENTION

The detailed description and preferred embodiments of the invention will be set forth in the following content, and provided for people skilled in the art to understand the characteristics of the invention.

The present invention is made based on the discovery that a RIP1 inhibitor and a MLKL inhibitor can increase the trans-epithelial electrical resistance (TEER) of POMGNT1 knockout human retinal pigment epithelial (hRPE) cells to an extent equal to that of wild hRPE cells.

Based on that, an embodiment of the present invention discloses a method for treating or preventing hereditary retinal dystrophy, and the method comprises: administering a composition comprising a RIP1 inhibitor or a MLKL inhibitor to a subject in need thereof.

The term “RIP1 inhibitor” used herein indicates a molecule that can efficiently inhibit the activity (phosphorylation) of RIP1 or can efficiently block the interaction of RIP1 with RIP3, and the example is, but not limited to, RIPA-56. RIPA-56 may be represented with the following chemical formula:

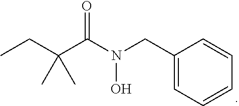 [SMILES: CCC(C)(C)C(=O)N(O)Cc1ccccc1]

The term “MLKL inhibitor” used herein indicates a molecule that can efficiently inhibit the activity (phosphorylation) of MLKL or can efficiently block the interaction of MLKL with RIP1 or RIP3, and the example is, but not limited to, GW806742X. GW806742X may be represented with the following chemical formula:

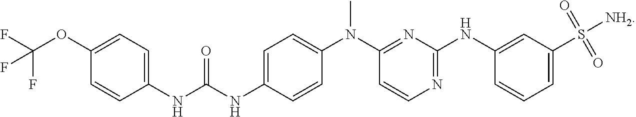 [SMILES: CN(c1ccc(NC(=O)Nc2ccc(OC(F)(F)F)cc2)cc1)c1ccnc(Nc2cccc(S(N)(=O)=O)c2)n1]

The term “hereditary retinal dystrophy” used herein indicates a genetic retinal disorder exhibiting both genetic heterogeneity and phenotypic heterogeneity. In term of gene, the example is, but not limited to, hereditary retinal dystrophy caused by Pomgnt1 mutation, such as hereditary retinal dystrophy caused by Pomgnt1^{L120R/L120R} mutation; in term of symptom, the example is, but not limited to, retinitis pigmentosa, Leber's congenital amaurosis, Usher syndrome, choroideremia, retinoschisis, or Stargardt disease.

The term “treating” used herein indicates actions to alleviate, ameliorate or relieve the symptoms of hereditary retinal dystrophy by administering the composition; the term “preventing” used herein indicates actions to inhibit or delay the occurrence of hereditary retinal dystrophy by administering the composition. That is, the treatment or the prevention can be achieved by administering the composition to the subject. Specifically, the treatment or the prevention can be achieved through inhibiting RIP-1 or MLKL by administering the composition to the subject. More specifically, the treatment or the prevention can be achieved through lowering the expression level of beclin1, P62, or LC3B or improving the TEER of retinal pigment epithelial cells by administering the composition to the subject. The example of the subject is, but not limited to, a mammal, the example of the mammal is, but not limited to, a primate, a cat, a dog, a rat, a mouse, a rabbit, a cow, a horse, a goat, a sheep, or a pig, and the example of the primate is, but not limited to, a chimpanzee, a human, a gorilla, a bonobo, an orangutan, or a monkey.

The term “administering” used herein indicates a suitable way to introduce the composition into the subject, and the example is, but not limited to, orally administering, sublingually administering, intrarectally administering, intranasally administering, intravaginally administering, intraperitoneally administering, percutaneously administering, epidermally administering, intraarticularly administering, intraocularly administering, or topically administering. For various administration routes, the composition is optionally in a suitable dosage form, which is, but not limited to, a tablet, a capsule, a granule, a solution, an emulsion, a suppository, a patch, an eye drop, an implant, or a powder.

Additionally, during the composition administering, gene therapy may be performed together. The gene therapy may be performed according to the gene causing hereditary retinal dystrophy. For example, a nucleic acid expressing protein POMGNT1 may be administered for hereditary retinal dystrophy caused by Pomgnt1^{L120R/L120R} mutation. Other examples can be reasonably obtained according to the foregoing instruction, and there is no need for detail.

The following examples are provided to exemplarily illustrate the present invention:

### Example 1: Pomgnt1^{L120R/L120R} Mice Produce Abnormal Electroretinogram (ERG) Responses

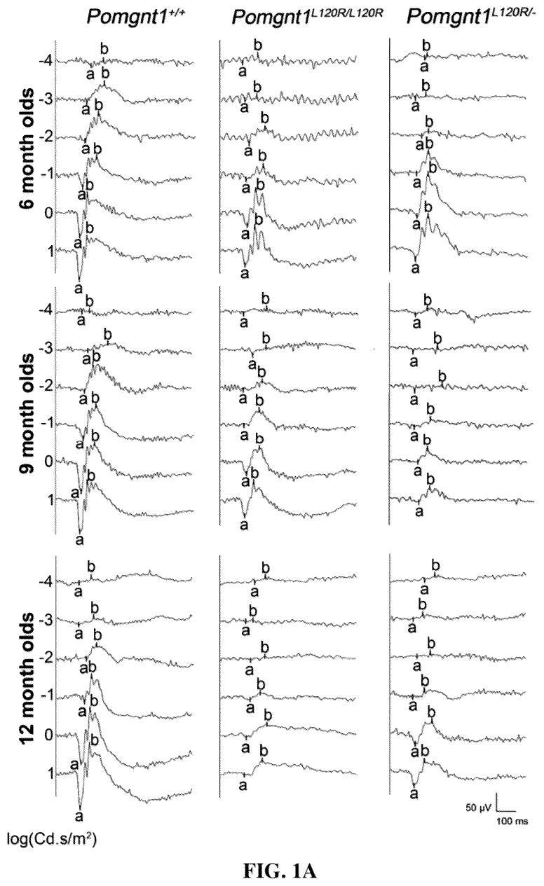

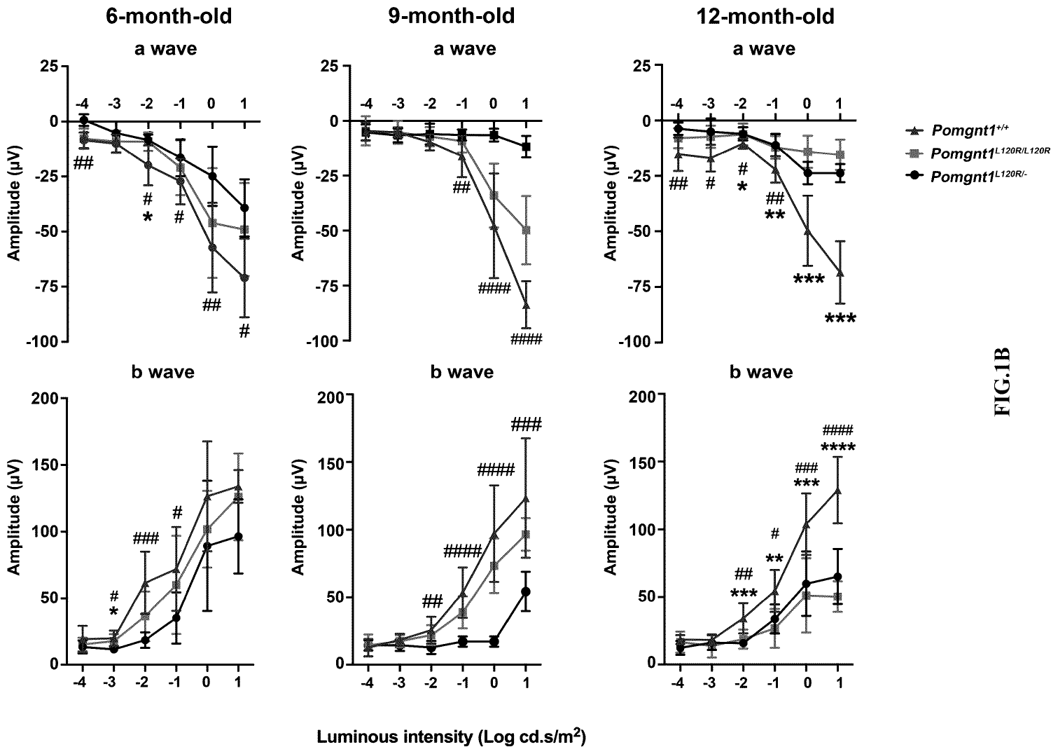

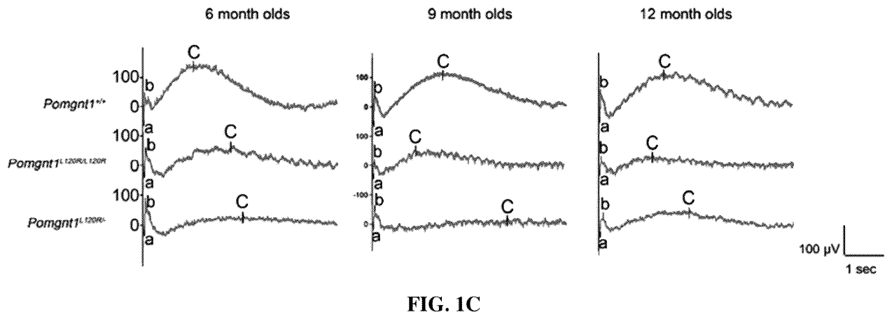

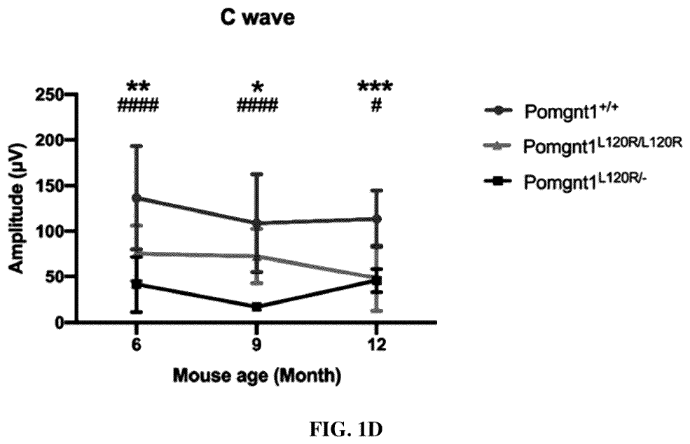

Patients with POMGnT1 homozygous L120R mutation displayed reduced or non-detectable b-waves in scotopic ERG examination but without mental retardation, muscle weakness, or atrophy. Hindlimb extension test showed that there was no muscle weakness or atrophy observed in the Pomgnt1^{L120R/L120R} mice. To evaluate retinal function in vivo, ERG was recorded with various light stimulus intensities under dark-adapted scotopic condition. Scotopic ERG records rod photoreceptor (a-wave), signal from rod to rod bipolar cell (b-wave), and retinal pigment epithelium (c-wave). Scotopic ERG intensity series at a range of light intensities (0.0001 to 10 cds/m²) recordings showed a dysfunction of rod and RPE. As shown in FIGS. 1A to 1D, a progressive decline in the Pomgnt1^{L120R/L120R} a-, b-, and c-wave amplitudes was observed in all three-age group (6, 9, and 12 months). This reduction in ERG a-, b, and c-wave amplitude indicated the function of rod photoreceptor, rod bipolar cells post-synaptic signal and RPE were greatly affected in Pomgnt1^{L120R/L120R} mouse retina.

### Example 2: Improvement of Electrophysiological Phenotypes Following AAV8-Human POMGNT1-GFP and RIPA-56 Treatment in Pomgnt1^{L120R/L120R} Mice

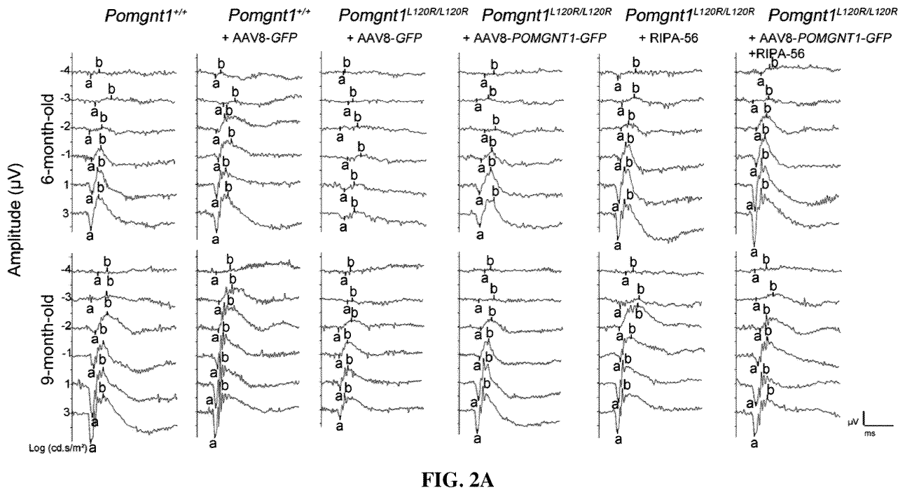

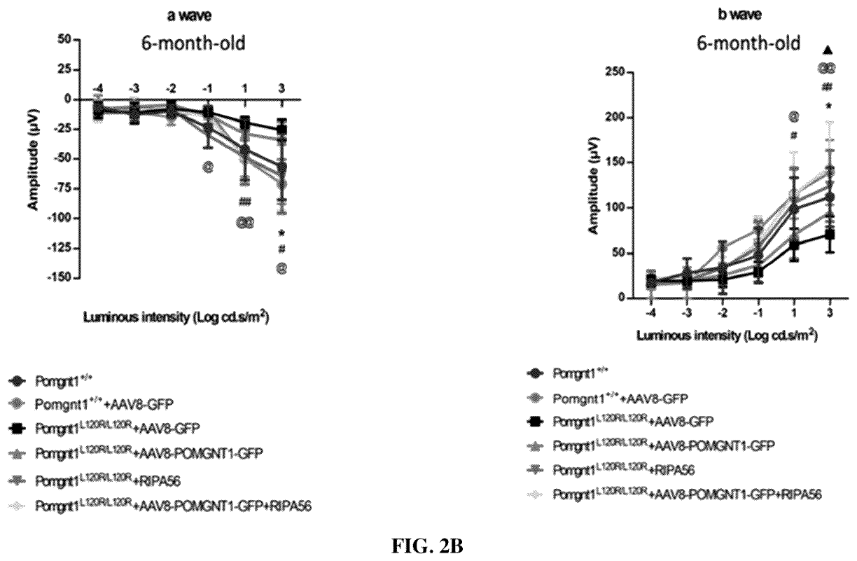

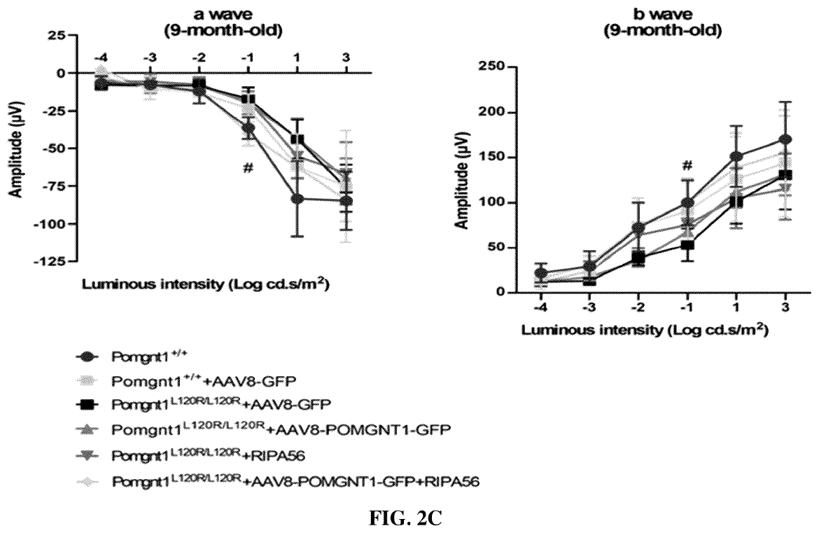

Three-month-old Pomgnt1^{L120R/L120R} mice and age-matched WT mice were injected with either AAV8-GFP (1 μL 1×10⁹ vector genomes (vg) subretinal injection), AAV8-hPOMGNT1-GFP (1 μL 1×10⁹ vector genomes (vg) subretinal injection), RIPA-56 (10 mg/Kg, twice a week, IP injection), AAV8-hPOMGNT1-GFP combined with RIPA-56 and evaluated 3 months, 6 months after injection. Scotopic ERG examination in 6-month-old mice (3 months after injection) showed significantly improved a-, b-, and c-wave amplitudes in AAV8-hPOMGNT1-GFP, RIPA-56 and AAV8-hPOMGNT1-GFP combined with RIPA-56 injection compared with that of AAV8-GFP treated group (FIGS. 2A and 2B). The amplitude of the a- and b-wave in the AAV8-hPOMGNT1-GFP combined with RIPA 56 treatment group was approaching WT values at the same age. Further, the scotopic ERG was performed in 9-month-old mice (6 months after injection) and also improved in the treated mice compared with AAV8-GFP-treated controls (FIGS. 2A and 2C). The results indicated single injection of AAV8-hPOMGNT1-GFP can efficiently improve electrophysiological phenotypes in Pomgnt1^{L120R/L120R} mice 6 months after injection.

### Example 3: Necroptosis is a Major Contributor to Retinal Degeneration in Pomgnt1^{L120R/L120R} Mice

S-arrestin (Sag) plays an important role in desensitization of rhodopsin, a G-protein-coupled receptor, to quench light-activated phototransduction in rod photoreceptor. Sag has also been found to participate in light-induced photoreceptor apoptosis through the formation of stable rhodopsin-arrestin complex through clathrin-dependent endocytosis. Furthermore, Sag has also been identified as an autoantigen which triggers immune responses in patients with uveitis.

Enolase 1 (Eno1) is a glycolytic cytoplasmic enzyme ubiquitously expressed in a variety of tissues. The interaction of Sag with Eno1 has been shown to reduce the catalytic activity of Eno1 by ˜25%. Photoreceptors are ranked as one of the highest energy-consuming cells in the body and they consume 10⁸ ATP/s/cell in the dark-adapted conditions. The alteration in catabolic efficiency of aerobic glycolysis could have a large impact on photoreceptors. Accordingly, the hypothesis is tested that L120R mutant enhances and stabilizes the Sag-Eno1 complex to further reduce the glycolytic activity and trigger inflammatory response in the retina. Subsequent energy deprivation and inflammation can activate necroptosis pathway.

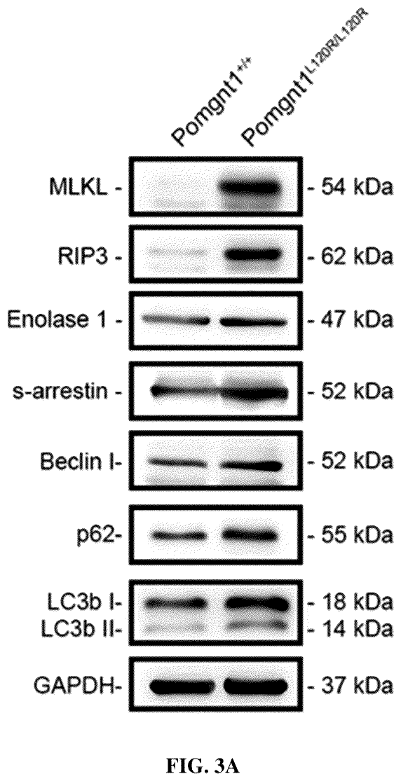

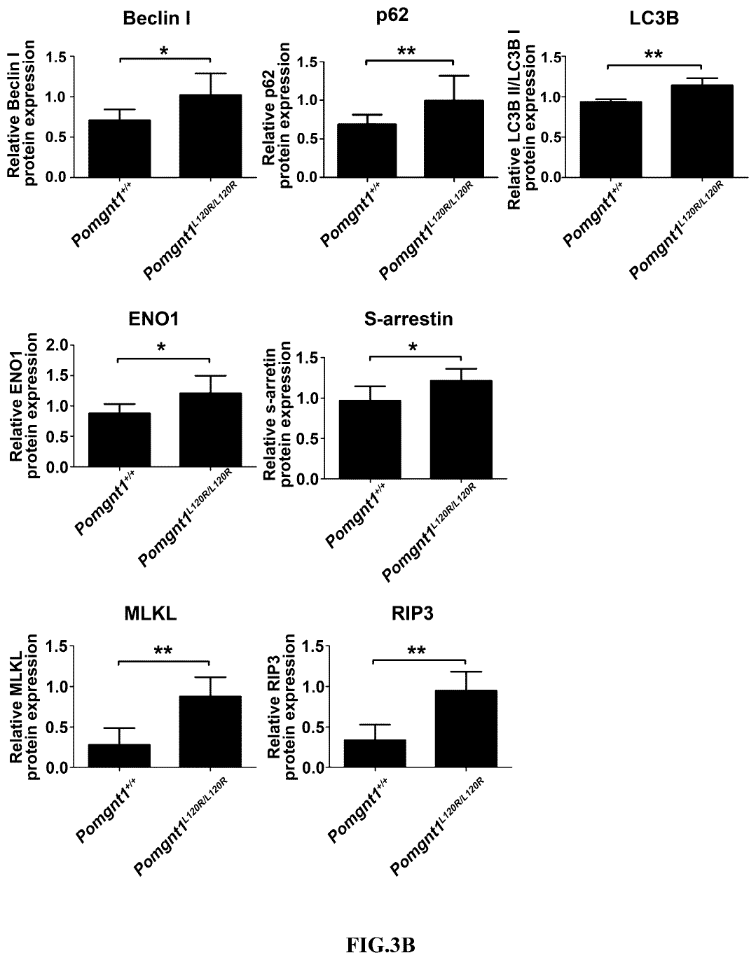

Eno1 and Sag protein expression levels were upregulated in Pomgnt1^{L120R/L120R} retina (FIGS. 3A and 3B). The expression level of autophagy-related proteins, beclin1, P62 and LC3B were enhanced in Pomgnt1^{L120R/L120R} retina, which indicates the inhibition of autophagic flux. RIP3 and MLKL protein expression levels were at low level in the WT retina but increased markedly in the Pomgnt1^{L120R/L120R} retina. RIP3 is a key regulator in necroptosis and its expression level has been shown to correlate with necroptosis. MLKL is a central downstream effect of RIP3 in the necroptotic pathway. The results of IHC staining and immunoblotting indicated that necroptotic mediated signaling is an important driver of retinal neurons death in Pomgnt1^{L120R/L120R} model.

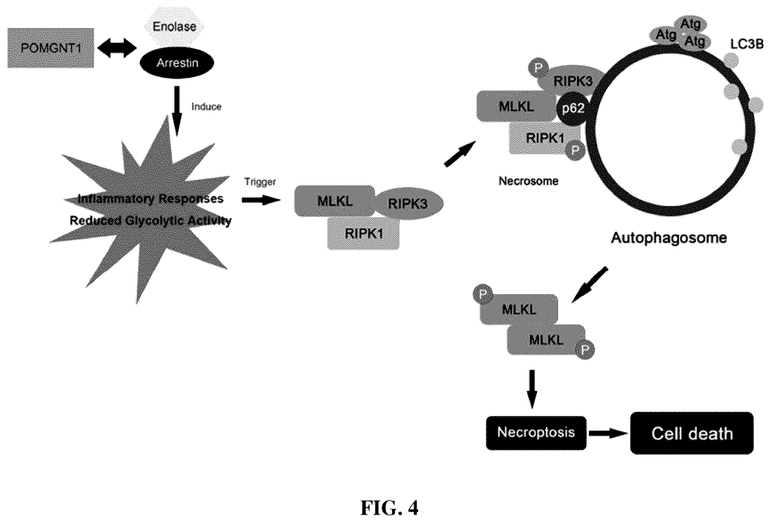

Based on these finding, a model is proposed to understanding the neuron death mechanism of in Pomgnt1^{L120R/L120R} model (FIG. 4). According to this model, L120R mutant protein may enhance and stabilize the Sag-Eno1 complex to trigger inflammatory response and reduce glycolytic activity in the retina. Subsequent ATP decrease and persistent inflammation further activate RIP3 and MLKL and inhibit autophagic influx leading to necroptotic death of retinal neuron cells.

### Example 4: Develop Potential Therapeutic Strategies

### 1. AAV-Mediated Gene Transfer of Pomgnt1 in Pomgnt1 Knockout Human RPE Cells

A plasmid encoding the human Pomgnt1 coding sequence was commercially purchased (GeneCopeia, Inc. Rockville, Maryland, USA) and packaged into a serotype 8 AAV vector. This plasmid contained the EF1a promoter and the coding sequence for enhanced green fluorescent protein (eGFP) fused downstream to Pomgnt1 (AAV8-Pomgnt1-eGFP). A null vector, containing identical regulatory sequences including eGFP but without Pomgnt1, was used as a control (AAV8-eGFP).

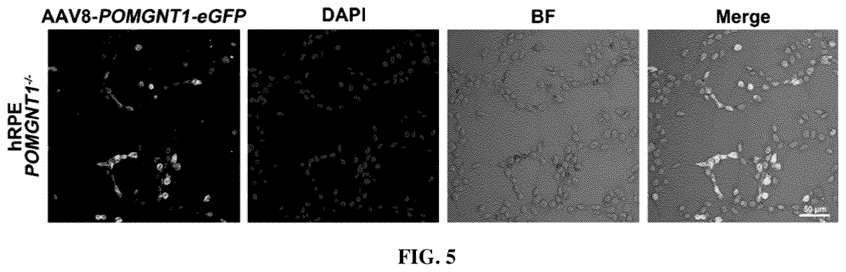

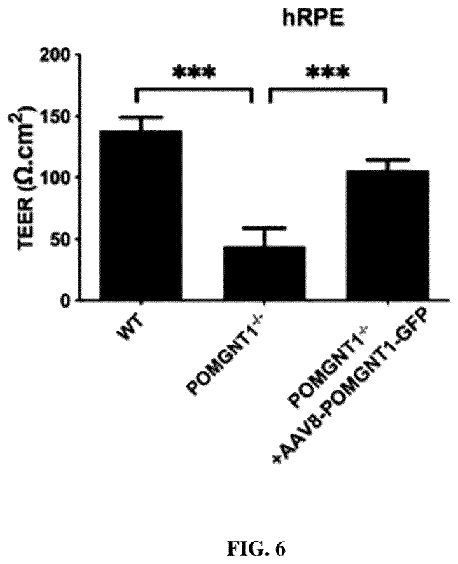

Immunocytochemistry of eGFP in POMGNT1 knockout human RPE cells after AAV transduction shows qualitatively higher eGFP expression cells (FIG. 5). The RPE is a polarized epithelial monolayer which lies between the photoreceptors and the choroid. The RPE is essential to keep neural retinal healthy by maintenance of many important functions including light absorption, trans-epithelial transport, phagocytosis, and secretion of growth factors. The feature of healthy RPE is the generation of a trans-epithelial electrical resistance by specific tight junctions between epithelial cells. Trans-epithelial electrical resistance (TEER) was determined in RPE monolayers in WT and POMGNT1 knockout cells using Millicell-ERS Voltmeter (MERS00002, EMD Millipore) with transwell culture to evaluate the cell monolayer integrity. As shown in FIG. 6, TEER level was significantly reduced in POMGNT1 knockout cells compared with WT. A significant improvement of TEER level was observed in POMGNT1 knockout cells after AAV-POMGNT1-eGFP transduction.

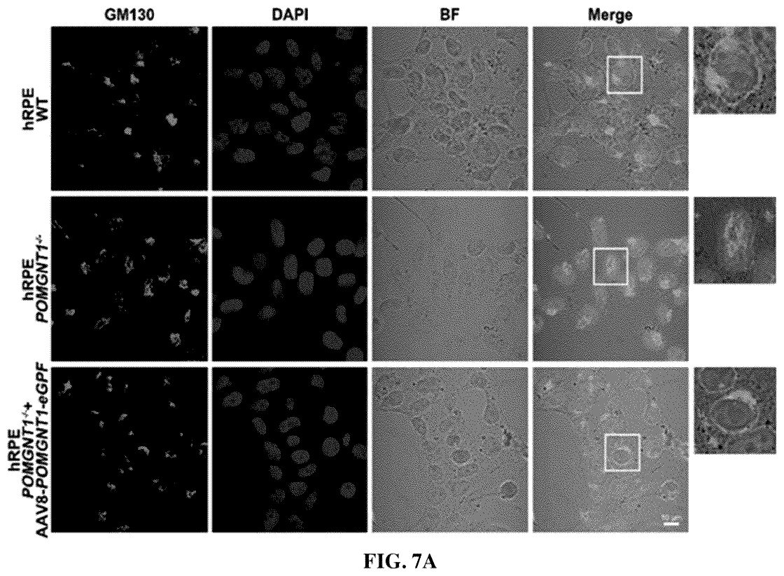

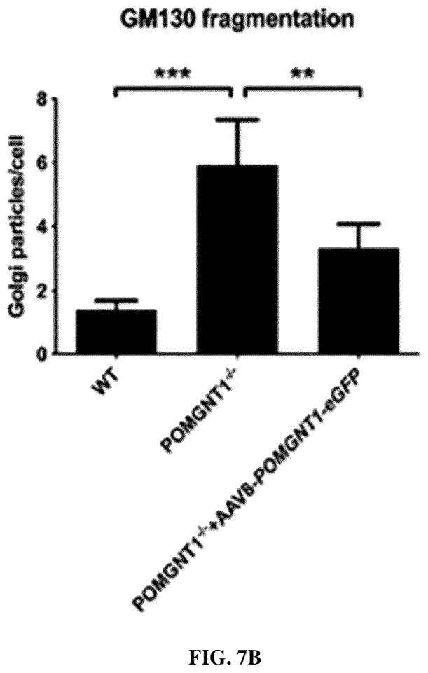

The Golgi complex (GC) consist of one or more stacks of flattened cisternae mainly distributed around the nucleus. During the progress of neurodegeneration, GC is broken into small isolated elements. GC fragmentation is an early event in many neurodegenerative diseases. The confocal microscopy images revealed that widely distributed GM130 staining small dots were observed in POMGNT1 knockout cells. Transduction of AAV8-POMGNT1-eGFP significantly reduced GC fragmentation in POMGNT1-depleted cells (FIGS. 7A and 7B).

### 2. The Effects of RIP1 (RIPA-56) and MLKL (GW806742) Inhibitor in Pomgnt1 Knockout Human RPE Cells

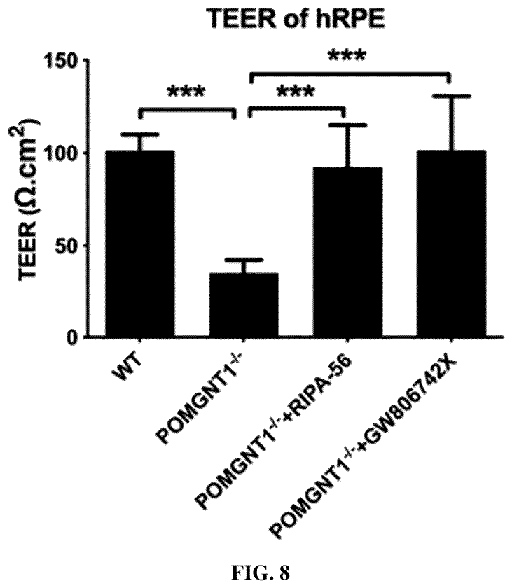

The previously-mentioned results indicate necroptosis is a major contributor to retinal degeneration in Pomgnt1^{L120R/L120R} mice. POMGNT1 knockout human RPE cell were treated with RIPA-56, a potent, selective RIP1 inhibitor or with GW806742X, a MLKL inhibitor. As shown in FIG. 8, significant improvement of TEER levels was observed in POMGNT1 knockout cells after RIPA-56 or GW806742X inhibitor treatment.

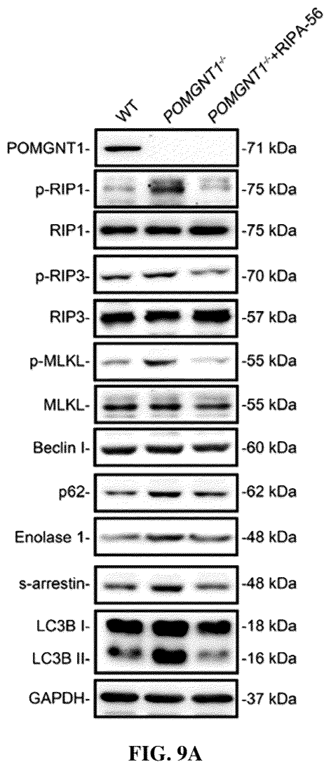

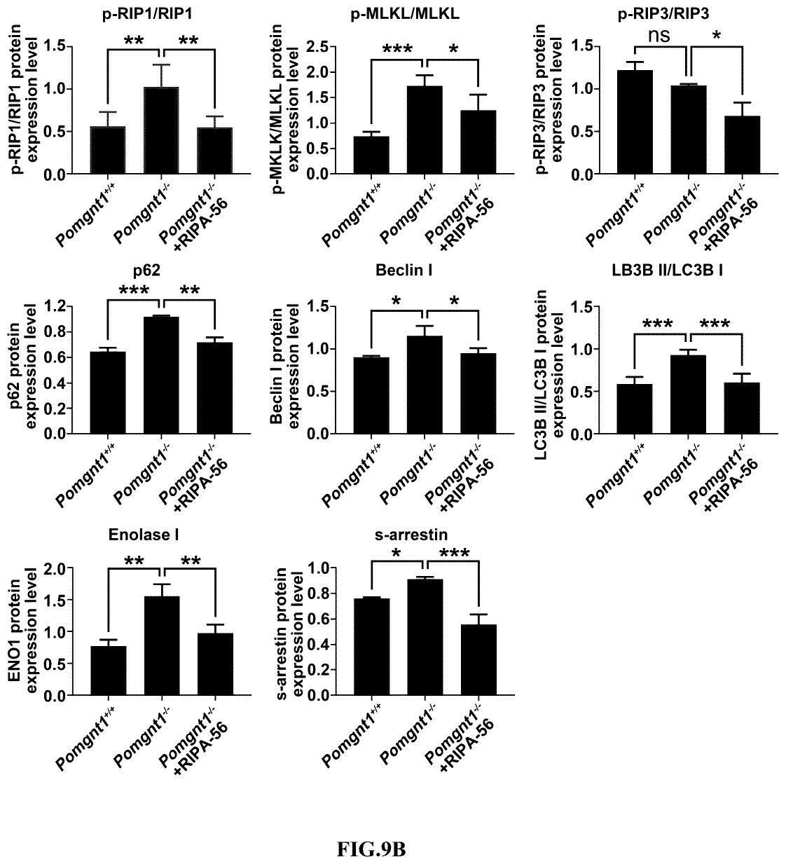

FIGS. 3A and 3B showed that the protein expression level of autophagy-related protein beclin1, p62 and LC3B were enhanced in Pomgnt1^{L120R/L120R} retina, which indicated that inhibition of autophagic flux. It was demonstrated that the necroptotic mediated signaling is an important driver of retinal neuron death in Pomgnt1^{L120R/L120R} mice model. The immunoblotting analysis of POMGNT1 knockout human RPE cell also showed significantly increased the expression level of beclin1, p62 and ratio of LC3BII/LC3BI and activation of necroptosis markers, phopho-RIP3 and phosphor-MLKL (FIGS. 9A and 9B). Treated with RIPA-56 can significantly decrease beclin1, p62 and LC3b and inhibit the phospho-RIP3 and phosphor-MLKL to improve the TEER level in POMGNT1 knockout human RPE cells.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for treating or preventing hereditary retinal dystrophy, comprising:
administering a composition comprising a RIP1 inhibitor or a MLKL inhibitor to a subject in need thereof.

2. The method as claimed in claim 1, wherein the RIP1 inhibitor is RIPA-56.

3. The method as claimed in claim 1, wherein the MLKL inhibitor is GW806742X.

4. The method as claimed in claim 1, wherein the hereditary retinal dystrophy is hereditary retinal dystrophy caused by Pomgnt1 mutation.

5. The method as claimed in claim 4, wherein the hereditary retinal dystrophy caused by Pomgnt1 mutation is hereditary retinal dystrophy caused by Pomgnt1^{L120R/L120R} mutation.

6. The method as claimed in claim 1, wherein the hereditary retinal dystrophy comprises: retinitis pigmentosa, Leber's congenital amaurosis, Usher syndrome, choroideremia, retinoschisis, or Stargardt disease.

7. The method as claimed in claim 1, wherein the composition is provided for lowering an expression level of beclin1, P62, or LC3B.

8. The method as claimed in claim 1, wherein the composition is provided for enhancing a trans-epithelial electrical resistance of retinal pigment epithelial cells.

9. The method as claimed in claim 1, wherein the composition administering step is performed in combination with gene therapy.

10. The method as claimed in claim 9, wherein the gene therapy comprises:
administering a nucleic acid for expressing protein POMGNT1 to the subject.

11. The method as claimed in claim 4, wherein the RIP1 inhibitor is RIPA-56.

12. The method as claimed in claim 4, wherein the MLKL inhibitor is GW806742X.

13. The method as claimed in claim 5, wherein the RIP1 inhibitor is RIPA-56.

14. The method as claimed in claim 5, wherein the MLKL inhibitor is GW806742X.

15. The method as claimed in claim 13, wherein the composition administering step is performed in combination with gene therapy.

16. The method as claimed in claim 15, wherein the gene therapy comprises:
administering a nucleic acid for expressing protein POMGNT1 to the subject.

17. The method as claimed in claim 14, wherein the composition administering step is performed in combination with gene therapy.

18. The method as claimed in claim 17, wherein the gene therapy comprises:
administering a nucleic acid for expressing protein POMGNT1 to the subject.
